(19) United States
(12) Patent Application Publication
(10) Pub. No.: US 2004/0102674 A1
(43) Pub. Date: May 27, 2004

(54) Minimally invasive percutaneous ventricular assist device

(76) Inventors: Filiberto P. Zadini, Camarillo, CA (US); Giorgio Zadini, Camarillo, CA (US)

Correspondence Address: FILIBERTO P. ZADINI, NORTH HILLS CALIF, 2237 HILLTOP LN, CAMARILLO, CA, 93012, US

(21) Appl. No.: 10/305,814

(22) Filed: Nov. 26, 2002

(51) Int. Cl. A61N 1/362

(52) U.S. Cl. 600/16

(57) ABSTRACT

A Minimally Invasive Ventricular Assist Device for circulatory support of a failing heart apt to be inserted percutaneously bluntly via a thoracostomy a into a chest cavity of a patient in cardiac failure or cardiac arrest without the need for wide surgical opening of the chest.

The device once deployed inside the chest is capable of compressing and decompressing the heart via pneumatic or mechanical means providing circulatory support to patients in cardiac failure or cardiac arrest.

Due to its design and structure the Minimally Invasive Percutaneous Ventricular Assist Device can be deployed rapidly and safely at bedside by health care professionals properly trained in the procedure.

### FIELD OF THE INVENTION

This invention relates to rapidly deployable devices for improving cardiac output in patients in cardiac failure or in cardiac arrest.

Background—Description of the Prior Art

Ventricular Assist Devices are designed to provide temporary or permanent support of the circulation in patients with reversible or irreversible cardiac failure. Various type of Ventricular Assist Devices have been designed to assist cardiac output in patient in cardiac failure or to create cardiac output in patients in cardiac arrest. In one type of Ventricular Assist Devices disclosed in US issued patents: U.S. Pat. No. 4,536,893, “Implant Device For Substaining The Activity Of The Myocardium” by Roberto Pallavicini; U.S. Pat. No. 3,279,464, “Myocardial Prosthetic Device” by Jacob Kline; U.S. Pat. No. 3,053,249 “Cardiac Massage Apparatus” by Edward Smith U.S. Pat. No. 4,957,477, “Heart Assist Jacket And Method Of Using It” by Stig S. Lundback; U.S. Pat. No. 3,478,737 “Heart Massager” by William Rassman, U.S. Pat. No. 4,048,990 “Heart Massage Apparatus”; U.S. Pat. No. 3,034,501 “Inflatable Heart Massager” by Carl E. Hewson; U.S. Pat. No. 4,048,990 “Heart Massage Apparatus” by Robert H. Goetz; U.S. Pat. No. 2,826,193 “Cardiac Resuscitation Device” by Arthur Vineberg; U.S. Pat. No. 5,131,905 “External Cardiac Device” by Ronald K. Grooters; U.S. Pat. No. 5,169,381 “Ventricular Assist Device” by Rober v. Snyders; U.S. Pat. No. 4,192,293 “Cardiac Assist Device” by Manfred Asrican; U.S. Pat. No. 3,455,298 “Instrument For Direct Mechanical Cardiac Massage” by George L. Anstadt, the heart is variously enveloped by a rigid or flexible but inextensible jacket, either one housing an inflatable/deflatable cuff or bladder which surrounds/envelops the heart, the cuff being connected to an exterior pneumatic source. Direct mechanical ventricular compression simulating the systole is achieved by the inflation of the cuff or bladder within the rigid casing or inextensible jacket while mechanical decompression of the ventricles simulating diastolic refilling of the heart is achieved by deflation of the cuff or bladder. Expansion and contraction of the cuff or bladder encircling the heart is also achieved beside pneumatic means by hydraulic means.

In other types of devices disclosed in U.S. Pat. No. 5,098,369 “Biocompatible Ventricular Assist And Arrythmia Control Device Including Cardiac Compression Pad and Compression Assembly” by Marlin S. Heilman; U.S. Pat. No. 4,506,658 “Pericardial Circulatory Assistance Device” by Jean P. Casile, compression of the ventricles is achieved mechanically by pads engaging the ventricular surface, cyclically actuated so as the ventricles are first compressed to aid blood ejection and second released to permit refilling.

Many investigators including Anstadt and Rassman have shown in their experiments that Ventricular Assist Devices are capable of generating pulsatile blood flow and blood pressure near to physiological levels providing circulatory support in cases of failing hearts even for days with negligible complications. Medical literature references include: William Rassman and C Walton Lillehei: “Long term maintenance of total or assisted cardiac function by means of an intracorporeal cardiac compressor”, Vol. XIII Trans Amer. Soc. Artif. Int. Organs, 1967; Mark p. Anstadt, George L. Anstadt and James E. Lowe “Direct mechanical ventricular actuation: A review”, Resuscitation, 21(1991)7-23.

Regardless however of the type of Ventricular Assist Device used, all these devices, in order to be applied to the heart and in order to be deployed, require opening the chest of a patient via a major surgical incision called thoracotomy which includes ribs spreading and or rib dissecting or cutting the sternum, a type of thoracotomy called median sternotomy. None of these devices is minimally invasive or rapidly deployable at bedside.

Citing Rassman in his paper “Long term maintenance of total or assisted cardiac function by means of an intracorporeal cardiac compressor”, Vol. XIII Trans Amer. Soc. Artif. Int. Organs, 1967, “the first and foremost disadvantage in using a ventricular assist device is that the chest must be open to apply the assistance device and, at this time, also to remove it”

Also Anstadt in his landmark article: “Direct mechanical ventricular actuation: A review”, Resuscitation, 21(1991)7-23 cites the requirement of thoracotomy as the first disadvantage in his reported list of the potential disadvantages of Direct Mechanical Ventricular Assist Devices.

None of the known Ventricular Assist Devices can be applied without major thoracotomy. The fact that none of these devices are deployable without a major surgical procedure such as thoracotomy, greatly limits the usefulness and clinical indications of the Ventricular Assist Devices. In a large number of patients who could greatly benefit from a Cardiac Assist Device, the deployment of a Ventricular Assist Device is not possible. Particularly, the rapid deployment of a Ventricular Assist Device is not possible at bedside or at the scene of a massive cardiac failure event, such as in cases of cardiac arrest.

None of these devices can be deployed rapidly, safely, percutaneously at bedside or at the scene of a cardiac arrest via a very small incision in the chest wall, by a minimally invasive procedure called thoracostomy.

### BRIEF SUMMARY OF THE INVENTION

With the present invention applicants propose a simple solution to the problem of placing a Ventricular Assist Device into the chest of a patient, without the requirement of major thoracotomy, at bedside or even at the scene in patients in cardiac arrest by properly trained health professionals such as paramedics.

Applicants propose an improved modified Ventricular Assist device composed as for above mentioned devices of a jacket or a sac-like structure capable of enveloping the heart and of compressing and decompressing the heart simulating systolic ejection and diastolic refilling, the major difference from the prior art being that the applicants device, due to its design and structure, is safely placeable into the chest cavity of a patient adjacent to the heart and removable from the chest without the need for a major thoracotomy, but simply via a thoracostomy, i.e. a small incision in the chest wall or upper abdomen.

Applicants' Ventricular Assist Device is inserted into the chest of patient and is designed to be passed thorough the chest wall in a contracted status, properly positioned adjacent to the heart and then deployed. Once deployed its structure and design allows compression and decompression of the heart providing circulatory support to a patient with a failing heart or an arrested heart.

### OBJECT OF THE PRESENT INVENTION

It is an object of the present invention to provide a simple, rapidly deployable and effective means for circulatory support to patients with a failing heart or victim of cardiac arrest.

It is an object of the present invention to provide health care workers with a simple, minimally invasive, effective, rapidly deployable apparatus and method for improving cardiac output in patients with heart failure or cardiac arrest.

It is an object of the present invention to provide health care workers with a simple, effective apparatus and method, rapidly deployable via a minimally invasive procedure, to improve cardiac hemodynamics in any case in which the heart is incapable of meeting the body demand for adequate tissue perfusion.

It is an object of the present invention to provide health care workers with a device, which is very simple to deploy and use, and at bedside and at the scene of a cardiac arrest.

It is an object of the present invention to provide patients victim of a failing heart with a ventricular assistance device capable of improving cardiac output generating pulsatile forward blood flow for a sustained period of time allowing the heart to recover or providing adequate circulation for bridging to cardiac transplantation.

It is an object of the present invention to provide patients victim of massive myocardial infarction in cardiogenic shock in emergency department or intensive care units settings with a Ventricular Assist Device capable of mechanically supporting the circulation, such a device being apt to be deployed at bedside with a minimally invasive and safe procedure.

### DRAWING FIGURES

FIG. 1 is side view of the device at rest ready to be inserted in the chest cavity.

FIG. 2 is an enlarged cross sectional view of a detail of the device of FIG. 1.

FIG. 3 is a cross sectional view of a detail of the device of FIG. 1, precisely of the partially deployed inflatable jacket following its insertion into the chest cavity.

FIG. 4 is a cross sectional view of a detail of the device of FIG. 1, precisely of the fully deployed inflatable jacket in its diastolic status.

FIG. 5 is a cross sectional view of a detail of the device of FIG. 1, precisely of the fully deployed inflatable jacket in its systolic status.

### DETAILED DESCRIPTION OF THE DEVICE

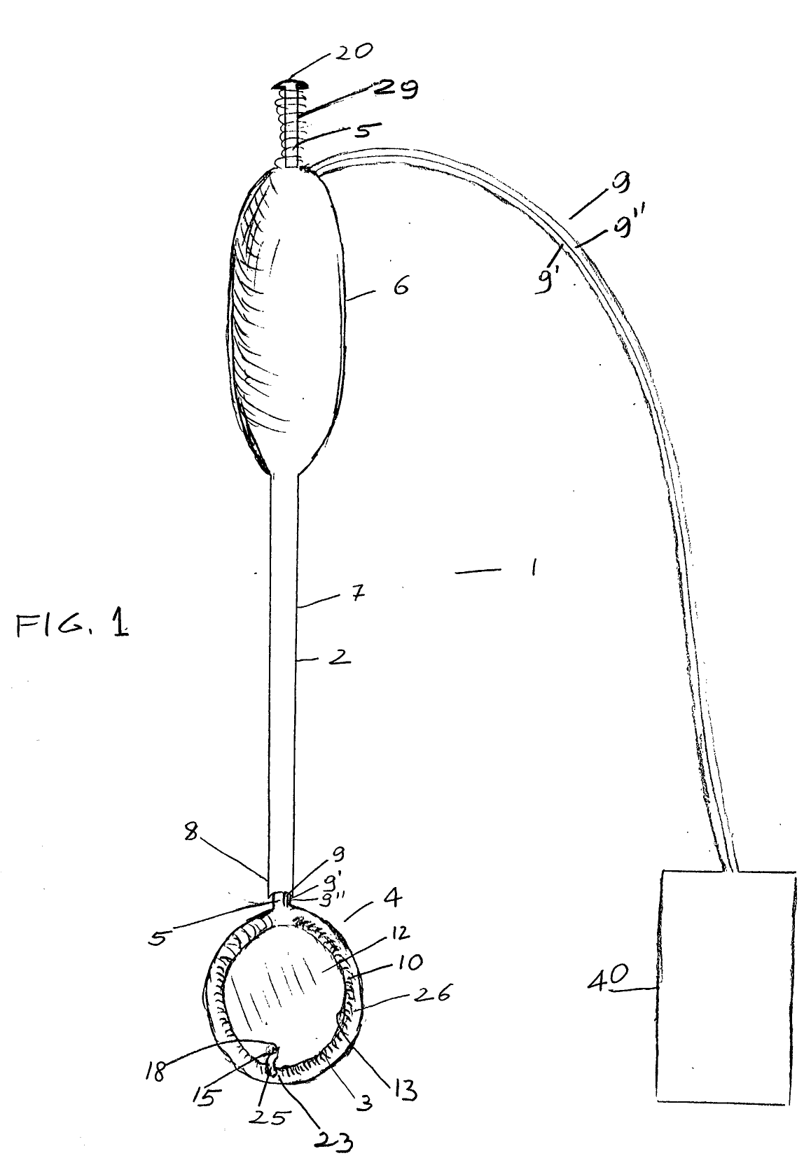

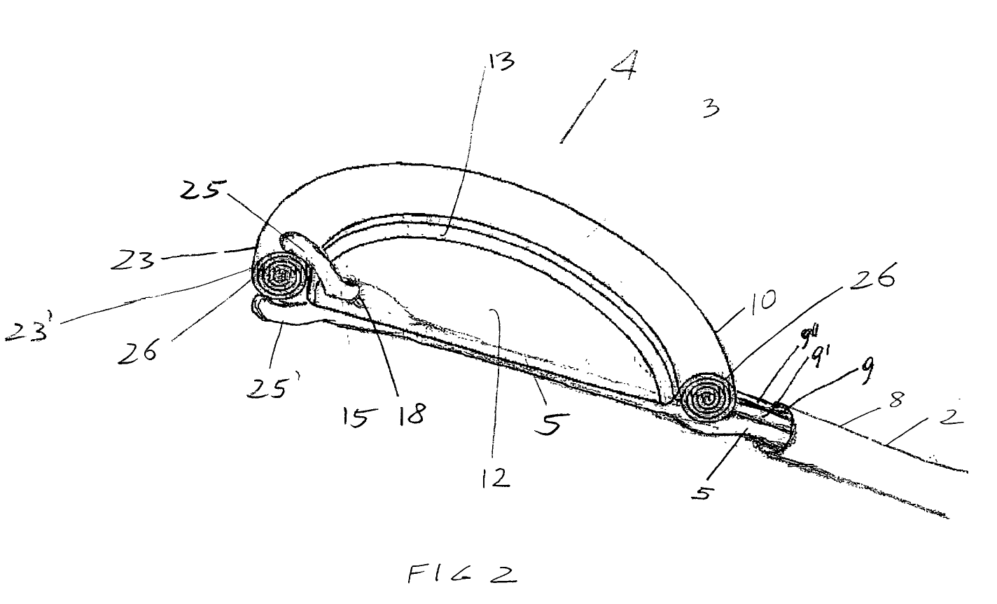

The device generally indicated at 1 is composed of a hollow stem 2 and heart compressing/decompressing member 4, which, in its packed contracted status as it is shown in FIGS. 1 and 2, prior to insertion into a chest cavity, appears generally donut shaped.

Hollow stem 2 may be made, although not necessarily, in rigid material and is firmly attached to compressing/decompressing member 4, which in FIGS. 1 and 2, being shown in its packed contracted status, appears donut shaped.

Compressing/decompressing member 4 is mainly composed of inflatable jacket member 10, which when fully deployed has the general appearance of a sac, and, as such, has a cul de sac or base or bottom membrane 12 and circular side walls 26. In its packed contracted status as it is better shown in FIG. 2, circular side walls 26 of jacket member 10 of compressing/decompressing member 4 are rolled outwardly down to cul de sac or bottom membrane 12. In such rolled down status, side walls 26 have, as mentioned above, the general appearance of a donut whose central opening is closed by cul de sac or bottom or base membrane 12. The donut shaped structure resulting from the rolling down of side walls 26 of jacket 10 is for sake of clarity designated with number 3 in FIGS. 1 and 2.

Hollow stem 2 is composed of handle 6, shaft 7 and has distal end 8.

Rigid rod 5 is slideably mounted within hollow stem 2, having proximal end 29 exiting from handle 6. Proximal end 29 is formed with button or flattened head 20, and spring 30 is mounted on rod 5 and interposed between handle 6 and button 20.

Hollow stem 2 houses doubled barrel tubular structure or double hose 9 formed with hose 9′ and 9″, double hose 9 connecting pneumatic source 40 to compressing/decompressing member 4 as it will explained below.

As shown in FIG. 2 which is an enlarged view of the compression/decompression member 4, rod 5 exits through open distal end 8 of stem 2 and is formed with distal end 15 grossly U-shaped having arms 25 and 25′ engaging and resting upon distal arc segment 23 of donut 3, formed of rolled down side walls 26 of heart compressing decompressing member 4. Rod 5 transverses membrane 12, arm 25 of distal end 15 exiting through hole 18 of base membrane 12 of jacket 10 as shown in FIG. 2.

As better shown in FIG. 5 where side walls 26 of inflatable jacket member 10 are unrolled up and compression/decompression member 4 is fully deployed, side walls 26 are triple layered, being formed with outer layer 14, made with flexible but substantially inextensible material, intermediate layer 14′, made also with flexible but substantially inextensible material, and inner layer 14″, made with compliant resilient material. Outer layer 14 and intermediate layer 14′ delimit narrow airtight compartment 35. Airtight compartment 35 is in airflow communication through connecting hose 9′ of double hose 9 with pneumatic source 40. Airtight compartment 35 is maintained narrow upon full inflation by a number of adhesions 50 between outer layer 14 and intermediate layer 14′. Such adhesions may engage layers 14 and 14′ in multiple points or multiple lines, such as vertical lines or horizontal lines or variously shaped areas. Outer layer 14 and intermediate layer 14′ are sealed together along free upper border or rim 16, and along rim 13 of side walls 26 of inflatable jacket 10. Rim 13 is tightly connected to cul de sac or base membrane 12, so that outer layer 14 and intermediate layer 14′ are in continuity with base membrane 12.

Rim 16 delimits wide mouth opening 17 as shown in FIG. 5.

Inner layer 14″ is sealed to intermediate layer 14′ along or in proximity of free border or rim 16 of side walls 26 of inflatable jacket 10 and is also sealed along or in proximity of rim 13 of side walls 26 of inflatable jacket 10.

Inner layer 14″ of inflatable jacket 10, together with intermediate layer 14′, delimit airtight compartment 36. Airtight compartment 36 is in airflow communication through connecting hose 9″ of double hose 9 with pneumatic source 40.

Compartment may be in turn subdivided in two sub compartments for independent compression/decompression of the two cardiac ventricles, in which case it will be required to double hose 9″ to connect each sub compartment to individual pneumatic sources.

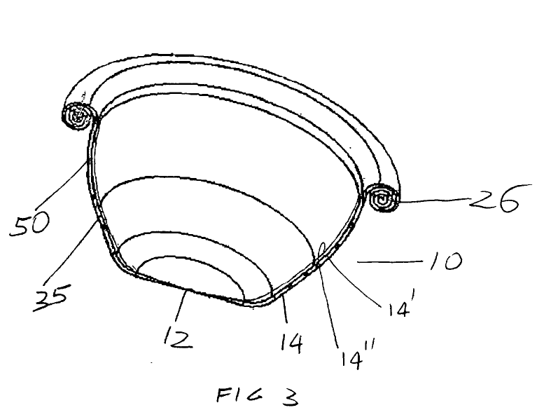

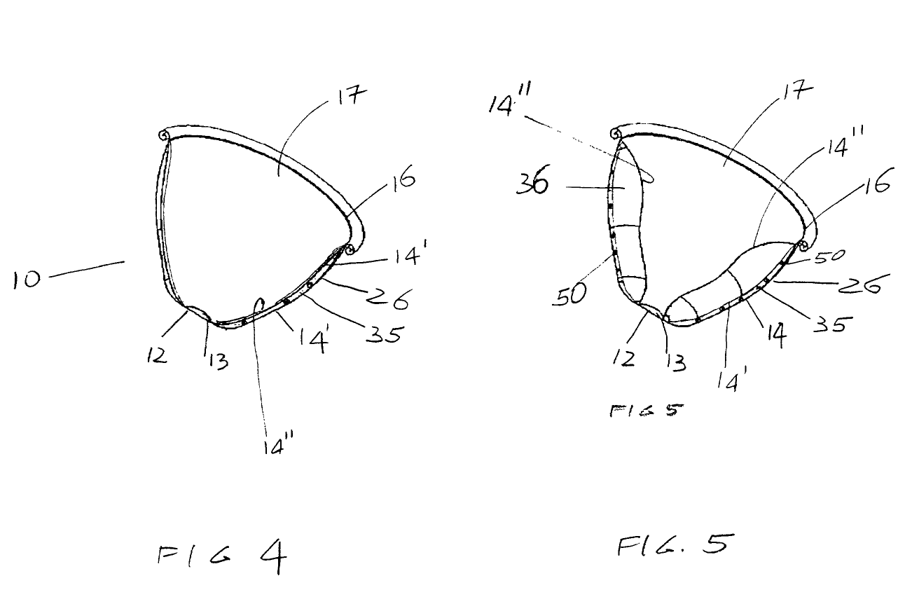

As better shown in FIGS. 3, 4 and 5, jacket member 10 when deployed inside the chest assumes grossly a conic shape with base membrane 12 contacting the heart apex and with sidewalls 26 contacting and enveloping the ventricles, rim 16 being positioned grossly at the atrioventricular junction of the heart when jacket member 10 is fully deployed.

Device 1 can be provided with at least one electrically conductive point to contact the heart surface for electrical cardiac therapy said point being connected to an external source of electrical activity such as a defibrillator.

In use, the operator makes with a surgical knife a small superficial skin incision of approximately one inch or less in length on the chest anterior wall in the fourth or fifth intercostals space, such incision being limited to the skin layer only. The operator grabs device 1 by handle 6, push down rigid rod 5 by applying pressure on flattened head or button 20 of rod 5. Distal end 15 of rod 5, being advanced in respect of hollow stem 2 by the operator pressing on button 20, and being engaged on distal arc segment 23 of donut 3, formed of rolled down side walls 26 of heart compressing decompressing member 4, will stretch compressing/decompressing donut 3 changing its shape from grossly circular to grossly elliptical, with a transverse diameter greatly reduced in respect of the greatly augmented longitudinal diameter. Segment 23 of donut 3 of compressing/decompressing member 4 becomes at this stage tip 23′ of the now elliptical shaped compressing/decompressing member 4. Tip 23′ of now elliptical shaped compressing/decompressing member 4 is placed into the skin opening created by the superficial skin incision in the chest wall and advanced by blunt dissection through the thickness of the chest wall for safe entry into the chest cavity until the whole compressing/decompressing donut like member 3 and part of stem 2 enters the chest cavity in front of the heart. The operator then manipulates device 1 acting upon handle 6 by tilting it in a way that stem 2 is nearly parallel to the chest wall and directing stem 2 carrying compressing/decompressing member 4 medially and caudally towards the heart apex.

The operator will release button 20 and donut member 3 will resume its original circular shape due to withdrawal of rod 5 in respect to hollow member 2, by spring 30.

By tilting stem 2 acting on handle 6, compressing/decompressing donut like member 3 will be advanced within the chest wall in the virtual space between the anterior chest wall and the heart until engagement of compressing/decompressing donut like member 3 with the apex of the heart will occur.

At this point pneumatic source 40 via hose 9′ will be actuated resulting in inflation of outer compartment 35 of jacket 10. As outer compartment 35 of jacket 10 inflates, side walls 26 of inflatable jacket 10, which, as seen above, have been inserted into the chest outwardly rolled down, will inwardly unroll under inflating pressure on the surface of the heart ventricles and, by unrolling, side walls 26 will gradually and quickly cover the surface of the heart ventricles from bottom up, at least up to the atrioventricular junction. The full inflation of compartment 35 will enable jacket member 10 to assume a sort of rigid shell configuration, perfectly equivalent to the rigid shell of most of currently used Ventricular Assist Devices.

At this point compressing/decompressing unit 4 is ready for alternatively compressing/decompressing the heart ventricles by intermittent inflation of compartment 36 of inflating jacket 10. Upon inflation of compartment 36, outside wall of compartment 36 which is nothing but intermediate layer 14′, will not displace outwardly due to the inextensibility of the material of intermediate layer 14′, while being made of compliant material, inner wall of compartment 36, which is nothing but inner layer 14″ of side walls 26 of jacket 10, will displace inward, toward the heart.

Inflation of compartment 36 will therefore result with uniform all around compression of the heart ventricles and consequent systolic ejection of blood from the ventricles.

Deflation of compartment 36 will allow decompression of the heart ventricles, with diastolic refilling of blood into the heart.

In order to favor decompression of the ventricles, inner layer 14″ of side walls 26 may be formed with a number of mini-suction cups formed from inner layer 14″ of side walls 26, such mini-suction cups maintaining adhesion to the surface to which they are applied by creation of vacuum within the mini-suction cup. Once layer 14″ is attached to the outer surface of the ventricles by means of such suckers, the resiliency of the material of layer 14″ will help decompression of the heart ventricles in the diastolic phase, when layer 14″, no longer pushed toward the heart by positive pressure inside compartment 36, is allowed to retract to its resting position. Other means, mainly based on vacuum, can be used to maintain adhesion between retracting layer 14″ and outer surface of the heart ventricles.

By cyclically inflating and deflating jacket 10, compression and decompression of the heart is achieved, systole and diastole are artificially reproduced and circulatory support to a failing or arrested heart is maintained or created ex novo.

Once the clinical status of the patient in cardiac failure is stabilized, jacket 10 is completely deflated resuming its contracted status and the device can be extracted through the thorocostoma created at time of insertion.

If it becomes necessary, for freeing the apex of the heart from the diaphragm a pericardiotomy can be performed to engage the compression decompression member with the heart apex.

Device 1 can be applied to the heart alternatively not only through a hole in the chest wall, but through a hole in the upper abdomen, in the subxyphoidea region just below the xyphoid process of the sternum, exactly with the same procedure used to create a hole in the chest. Device 1 will be advanced by blunt dissection for safe entry into the chest cavity until donut shaped compression decompressing member 4 has engaged the heart apex. The rest of the operation is the same as above described.

If necessary, in order to ascertain or aid correct placement of device 1 in respect to the heart the operator can utilize a thoracoscope at bedside and manipulate handle 6 in order to guide the device to engage compression decompression member 4 with the heart apex.

What we claim is:

1. A ventricular assist device for circulatory support of a failing or arrested heart, comprising:
a heart compressing decompressing member, said member being capable of actively compressing the heart on at least two opposing surface of the heart, the compressing decompressing member being reducible in overall size to allow its passage into the chest cavity through a thoracostomy opening in the chest wall, said heart compressing decompressing member having at least one inflatable compartment, and
a tubular member in airflow communication with said inflatable compartment to enable inflation of said inflatable compartment by extrathoracic pneumatic sources.

2. The device of claim 1 further comprising an inflatable jacket insertable in a contracted status into the chest cavity, said inflatable jacket being, upon its placement in correspondence of the apex of the heart, self deployable on the surface of the heart upon inflation.

3. The device of claim 2, wherein said inflatable jacket has a base membrane and side walls, said side walls being rolled down to said base membrane to achieve said reduced size of said compressing decompressing member during its insertion through the chest wall.

4. A minimally invasive ventricular assist device for circulatory support of a failing heart, comprising:
A heart compressing decompressing member, said member being apt to apply pressure and release pressure on at least two opposed surfaces of the heart, and
a stem connected to said compressing decompressing member, to enable insertion of said compressing decompressing member via blunt dissection into a chest cavity through a hole formed in said chest cavity.

5. The device of claim 1 further comprising conductive electrical contacting means for the heart surface for electrical cardiac therapy.

6. The device of claim 4 wherein said heart compressing decompressing member is placeable into the chest cavity adjacent to the heart through an hole formed in a subxyphoidea region by blunt dissection.
